(19) United States
(12) Patent Application Publication
(10) Pub. No.: US 2012/0071414 A1
(43) Pub. Date: Mar. 22, 2012

(54) METHOD FOR PRODUCING AND USING ACTIVE PRINCIPLES OF LIMESTONE

(75) Inventors: Xavier Bourrat, Marcilly-En-Vilette (FR); Evelyne Lopez, Paris (FR); Marthe Rousseau, Vandoeuvre-Les-Nancy (FR)

(73) Assignees: Centre National De La Recherche Scientifique-CNRS-, Paris (FR); Universite D'Orleans, Orleans (FR); Museum National D'Histoire Naturelle, Paris (FR)

(21) Appl. No.: 13/262,909

(22) PCT Filed: Apr. 9, 2010
(86) PCT No.: PCT/FR10/00298

§ 371 Date: Nov. 21, 2011

(30) Foreign Application Priority Data
Apr. 10, 2009 (FR) 09/01804

(51) Int. Cl. A61K 8/64; A61K 31/70; A61K 31/20; C07K 1/14; C07H 1/08; C11B 1/10; A61P 19/00; A61P 19/02; A61P 19/08; A61P 19/10; A61P 17/00; A61Q 19/08; A61Q 19/00; A61K 8/60; A61K 8/36; A61K 38/02

(52) U.S. Cl. 514/16.9; 514/1.1; 514/16.7; 514/17.1; 514/18.6; 514/18.8; 514/23; 514/558; 530/422; 530/423; 530/424; 530/426; 536/127; 554/206; 554/210

(57) ABSTRACT

The present invention relates to a method for extracting organic molecules and/or organic minerals from a limestone material, including placing said limestone material in contact with a solution including at least one organic solvent, water and at least one acid, as well as to the organic molecules and/or organic minerals produced by the method. The products produced by the method can be used, for example, in therapeutic and non-therapeutic fields, in particular the agri-food, cosmetic and medical fields.

### TECHNICAL FIELD

The present invention relates to a method for extracting organic and/or organomineral molecules from a limestone material, and also to the extracts obtained by means of this method.

The products extracted are organic and/or organomineral molecules.

The method and the products obtained by means of this method can be used, in particular, in therapeutic and non-therapeutic fields: in particular medical, cosmetic and food-processing fields, in particular nutraceuticals, food supplements and/or medicinal foods.

In the description below, the references between parentheses (Ref) refer back to the list of references presented at the end of the text.

### PRIOR ART

Methods for extracting organic molecules from various sources are known in the prior art. They are, for example, methods for extracting organic molecules from plant extracts, for example extracts of Arachis hypogaea (Fabaceae) and of Phaseolus vulgaris (Fabaceae). The molecules extracted with these methods can be used in various fields, for example in the pharmaceutical industry, the cosmetics industry, in the food-processing field, etc. Examples of organic molecules extracted in the prior art are, for example, sterols, terpenes, terpene esters, etc. These molecules are, for example, used as food additives or in cosmetic products in order, in particular, to make up for deficiencies and/or to treat pathological conditions of the skin or the like.

Decreased bone mass and density is an important public health problem, more particularly in women after the menopause; it also occurs in men over 50 and more rarely in children.

Indeed, after the menopause, the decreased oestrogen level leads to a loss of bone mass and density.

Decreased bone density may also be due to pharmacological treatments, for example anti-inflammatory treatments based on corticosteroids which can lead to osteopenia that can go as far as osteoporosis.

The pathological condition associated with a loss of bone mass and density is osteoporosis, which leads to an excessive brittleness of the skeleton.

Osteoporosis can also be due to the acquisition of insufficient bone material at the end of growth, or to bone loss during adult life owing, for example, to genetic, nutritional and environmental factors. One of the means known in the prior art for preventing or treating decreased bone mass and density is generally the administration of calcium-rich diets. Various forms of calcium are used; it may, for example, be CaCO₃ (see J. L Greger et al. “Mineral utilisation by rats fed various commercially available calcium supplements or milk.” J Nutr; 117: 717-24, 1987 (Ref 1); T. Chevalley et al. “Effect of calcium supplements on femoral bone mineral density and vertebrate fracture in Vitamin D repleted elderly patients”, Osteoporosis Int 4: 245-52, 1984 (Ref 2)). Some works have studied the effect of the assimilation of powders based on coral (Blumberg S. “Is coral calcium a safe and effective supplement? ” J Am Diet Assoc September; 104(9): 1335-6 (Ref 3)), mother of pearl, fossil shells (T. Okano et al. “Bioavailability of calcium from oyster shell electrolysate and DL-Calcium lactate in vitamin D-replete or vitamin D-deficient rats.” Journal of Bone and Mineral Metabolism 11 (2): 23-32, 1993 (Ref 4); T. Miura “Calcium bioavailability of a total bone extract (TBE) and diets effects on bone metabolism in rats”, Biosci. Biotechnol. Biochem 62: 1307-12, 1998 (Ref 5)) or else eggshell (Y Takada “Bioavailability of various dietary calcium compounds. 2 Examination in vivo of various dietary compound”. Snow brand milk products Co, Ltd Sapporo: 75-84 (Ref 6); A Schaafsmal et al. “Eggshell powder, a comparable or better source of calcium than purified calcium carbonate: piglet studies” J Sci Food Agric 79: 1596-600, 1999 (Ref 7)). These various forms of “natural” calcium seem to show an “improved” assimilation.

However, these solutions are not sufficient since they do not make it possible to completely treat certain pathological bone conditions, in particular osteoporosis. Furthermore, the methods for extracting these molecules or producing them have low yields that therefore require the use of a large amount of raw materials and therefore high running costs.

Another means known in the prior art for treating osteopenia (osteoporosis) is the administration of anti-osteoporotic agents. These are, for example, compounds which stimulate bone formation and calcium binding: vitamin D, a parathyroid hormone (at a very low dose), estrogens and strontium; compounds which slow bone resorption; bisphosphonates and calcitonin. An intake of readily bioavailable calcium is still necessary during these treatments. It has been shown in rats that parathyroid hormone at high doses is carcinogenic. Moreover, vitamin D has effects on immunity which can be harmful to humans.

These products generally have not insignificant side effects and consequently can be used only over short periods of time with calcium supplementation. These products do not therefore make it possible to do away with concomitant calcium intakes. Excessive calcium intake can be a source of problems owing to ectopic fixations: kidney stones, aortic calcification etc. Osteoporosis can also affect animals, for example puppies between 2 and 6 months, old dogs which do not fix calcium, dogs fed exclusively on meat, and horses, for example horses that stay inside for too long.

However, none of the molecules known or used in the prior art make it possible to effectively treat decreased bone density in animals. Furthermore, the problems and side effects known for humans also occur in animals.

The methods known in the prior art that make it possible to extract the molecules used for, for example, the treatment of osteoporosis require large amounts of raw materials, for example of plants and have low extraction yields and therefore very high implementation costs.

A study carried out by the pharmaceutical industry has shown that close to two thirds of the active ingredients used in medicaments come from plants or are chemically copied from nature. Other molecules are therefore actively sought and, with them, methods for extracting them.

Moreover, calcium plays an important role in various functions of the dermal-epidermal junction (DEJ). It performs a structural role combined with the macromolecules that combine the basal layer to the collagen of the dermis. It is an important element in the cohesion between the dermis and the epidermis. The DEJ has a regulatory role, a role as a filter in the exchanges between the dermis and the epidermis; the epidermis is not vascularized, calcium is a key element in the communication and regulation mechanisms.

Moreover, calcium also has a role in the whole of the skin metabolism. This involves in particular terminal differentiation (TD) of the epidermis, where calcium is involved in keratinocyte differentiation. The calcium gradient increases from the germinative layer where the keratinocytes form toward the outer layers of the epidermis. The calcium binds to phospholipids, which are necessary for the keratinization process.

The products known in the prior art which make it possible to stimulate keratinization have many side effects, for example hypersensitization of the skin to ultraviolet rays.

Furthermore, the most important role of calcium in human physiology is that of maintaining calcium homeostasis (calcium blood level) and as a cell messenger.

Calcium is also an important element in the field of livestock feed. This involves, for example, feed for egg-laying poultry.

There is therefore a real need to find new molecules which overcome these deficiencies, drawbacks and obstacles of the prior art, it being possible for said molecules to be used, for example, for treating and/or preventing decreased bone mass and density, for stimulating the activity and/or restructuring of the skin, and dietary supplementation with, consequently, the maintaining of calcium homeostasis.

There is also a real need to find a method which makes it possible to extract these molecules from nature in order, for example, to reduce the extraction cost and to find new active ingredients that can be used in the biomedical field (medicine, pharmacy, parapharmacy, nutrition and the like) and/or in the cosmetics industry and/or in the food-processing field.

There is a real need to effectively supplement the feed of animals by discovering new active agents capable of promoting calcium assimilation and fixation thereof, and of reducing the costs of producing these molecules.

### DESCRIPTION OF THE INVENTION

The objective of the present invention is precisely to reply to these needs and drawbacks of the prior art by providing a method for extracting organic and/or organomineral molecules.

The method of the invention is a method for extracting organic and/or organomineral molecules from a limestone material, comprising the following step: bringing the limestone material into contact with a solution, said solution comprising: at least one organic solvent, water and at least one acid.

The inventors are the first to have discovered that limestone materials containing numerous organic and/or organomineral molecules accumulated during the formation of said materials have pharmaceutical and/or cosmetic properties. They have therefore developed a method for extracting these molecules in order to extract them and to use them. In addition, the inventors have shown that the molecules extracted are capable of stimulating the mechanisms of bone formation and mineralization and/or of stimulating the activity of epidermal and/or dermal cells.

In the present invention, the term “limestone material” is intended to mean any material comprising limestone. It may, for example, be a limestone rock.

Preferably, it may be any of the natural forms of limestone rock comprising at least 50% of calcium carbonate regardless of the common name and form. These rocks may, for example, be in solid, friable, powdery forms, or the like.

According to the invention, the limestone material may, for example, be a limestone rock from France, chosen from the group comprising a rock from Orgon. France, stone from Euville. France, from Saint Germain, France, and from Pagny sur Meuse, France.

Preferably, the limestone material may, for example, be a limestone rock chosen from the group comprising rock from Orgon. F-13660, stone from Euville. F-5520, from Saint Germain, F-55140 or from Pagny sur Meuse, F-55190 or else from Fleming, F-57830. The notation “F” indicates the origin France and the number indicates the postcode.

In the present invention, the limestone material may originate, for example, from the quarries: of Fontvieille. F-13990; of Salon de Provence, F-13300; of Orgon, F-13660; of Cassis, F-13260; of Maine-de Boixe, F-16230; of Biarge à Saint Fraigne, F-16140; of Gratte-Chat à Saint Sornin, F-17600; of Subdray. F-18570; of Sainte-Croix-De-Mareuil. F-24340; of Pont-du-Gard à Remoulin, F-30210; of Verfeuil, F-30630; of Conques à Brouzet-les-Ales. F-30055; of Val d'Epis, F-39160; Raymond Rabier, La Tieule, F-48500; of Senonville, Valbois F-55300; of Pagny-sur-Meuse, F-55190; of Euville. F-55200; of Saint Germain-sur-Meuse, F-55140; of Héming. F-57830; of Saint Maximin, F-60740; of Arancou, F-64270; of Rébenacq. F-64260; of Roquemaure à Orange, F-84100; of Chaux de la Tour à Coustellet. F-84220; of Menerbes. F-84560; of Estaillades à Oppede, F-84580; of Saint Maurice La Clouère, F-86160; and of Feriana, Tunisia.

According to the invention, the preferred limestone rock has a measurable total organic carbon (TOC) content, but it may just as easily be very low, or even undetectable. For example the TOC content may be between 0.01 and 1.5%, for example between 0.02 and 0.8% or, for example, between 0.03 and 0.3%.

The total organic carbon (TOC) content can be measured by any method known to those skilled in the art. It may involve, for example, Leco (registered trademark) analysis of the organic carbon of decarbonated rocks (for example Leco (registered trademark) SC-444) or else an elemental analysis of concentrated organic materials using a kerogenatron (Vinci Technologies, France) or else a pyrolytic method known as Rock Eval pyrolysis, which has been used here, diverted from its customary use, namely mineral oils or contaminations (Rock-Eval 6 turbo from Vinci Technologies, France; see E. Lafargue, F. Marquis, D. Pillot, “Rock-Eval 6 Applications in hydrocarbon exploration, production and soil contamination studies”, Oil & Gas Science and Technology, 1998, 53(4), 421-37 (Ref. 8)). For example, for very low concentrations of endogenous organic matrix, the criterion selected consists in recording the appearance in the limestone of an S2-type cracking peak which does not exist in a purely chemically precipitated carbonate (e.g. as supplied by VWR International SA). TOC analysis, like the use of Rock-Eval pyrolysis, are indirect methods which do not in any way determine the chemical nature of the molecules, or their activity.

According to the invention, the limestone material may, for example, have an average particle size of between 0.5 and 1000 μm, preferably between 0.5 and 500 μm, even more preferably less than 200 μm.

According to the invention, the expression “organic and/or organomineral molecules” is intended to mean, for example, proteins, peptides, lipids or saccharides or mixtures and/or, for example, molecules rich in heteroelements, for example a calcium binding protein, salts, ions, minerals, colloidal particles and/or combinations of these compounds. The organic and/or organomineral molecules may be in the complex form of the natural mixture, or separated and purified. They may, for example, be old molecules of which the age corresponds to the age of the limestone material or, for example, molecules related to diagenesis or formed over time, or in recent limestone.

According to the invention, in addition, the term “extract” or “extracts of rock” or alternatively “extract of limestone rock” is used to denote, in equivalent manner, the organic and/or organomineral molecules as defined above; for example, they may be proteins, peptides, lipids, saccharides and/or a mixture and/or for example salts, ions, minerals, colloidal particles and/or combinations of these compounds.

According to the invention, the term “organic solvent” is understood to be, for example, a solvent or a mixture of solvents, chosen from the group comprising C1 to C6 alcohols, ethers and hydrocarbons. For example, it may be a solvent chosen from the group comprising ethanol, diethyl ether, dichloromethane, dimethylformamide (DMF), dimethyl sulfoxide (DMSO), acetonitrile, or a mixture of these solvents. It may also be, for example, without prejudging their nature, any fluids in the supercritical or subcritical state, having characteristics equivalent to “organic solvents”, for example supercritical CO₂ or alternatively subcritical water.

According to the invention, the term “mixture of solvents” is understood to mean, for example, a mixture of at least two organic solvents, at least three organic solvents, at least four organic solvents or more. The mixture of solvents can be, for example, a mixture of C1 to C6 alcohols, a mixture of ethers, a mixture of hydrocarbons and/or a mixture of solvents chosen from the group comprising C1 to C6 alcohols, a mixture of ethers and a mixture of organic hydrocarbons or a mixture of these solvents.

According to the invention, any concentration of suitable solvents can be used. The concentration by volume of the organic solvent relative to the aqueous phase is advantageously between 1 and 99%.

In the present invention, the term “acid” is intended to mean, for example, an inorganic acid and/or an organic acid. For example, the acid may be chosen in the group comprising hydrochloric acid, sulfuric acid, acetic acid, citric acid and ethylenediaminetetraacetic acid.

Preferably, in the present invention, the pH of the solution of the method of the invention may be less than 7, preferably between 0 and 7, even more preferably between 0 and 5, more preferably between 1 and 4, more preferably between 1 and 3.

In the present invention, the bringing of the limestone material into contact with the solution can be carried out, for example, by dipping, sprinkling, wetting, or partial or total immersion of the limestone material with said solution.

Preferably, the bringing of the limestone material into contact with the solution is carried out by total immersion of the material.

According to the invention, the bringing of the limestone material into contact with the solution comprising: at least one organic solvent, water and at least one acid, can be carried out, for example, at a temperature between 5 and 90° C., preferably between 8 and 75° C., even more preferably between 8 and 30° C. This temperature can also be chosen within the entire range necessary for subcritical or supercritical states, if necessary. The temperature is generally chosen so as not to irreversibly denature the molecules extracted.

According to the invention, the bringing of the limestone material into contact with the solution comprising: at least one organic solvent, water and at least one acid, can be carried out, for example, for a period of time between approximately 1 minute and 72 hours, preferably between approximately 1 minute and 48 hours, even more preferably between 1 minute and 12 hours.

According to the invention, the method can advantageously also comprise a crushing step before the limestone material is brought into contact with the solution.

In the present invention, the term “crushing” is intended to mean fractionation of the rock via, for example, industrial mechanical means or via any type of crushing where the temperature is sufficiently low so as not to irreversibly denature the molecules extracted.

The crushing step can be carried out, for example, according to the techniques known to those skilled in the art for obtaining a limestone material with an average particle size described above. It may, for example, be crushing carried out by means of a technique chosen from the group comprising ball crushing, roller crushing, pestle/mortar crushing, hammer crushing, impact hammer crushing, swing-disk crushing, vibration crushing, dual toothed roll crushing and/or dual smooth roll crushing.

Preferably, the limestone material has a particle size of less than 200 μm. Indeed, this particle size can make it possible to optimize the extraction yield of the method of the invention.

According the invention, the method may also comprise a step of recovering the organic and/or organomineral molecules. It may be, for example, a filtration step. This step makes it possible to separate the limestone material from the solution in which the organic or organomineral molecules are extracted. It can be carried out, for example, by means of a filter, for example having pores with a diameter chosen in relation to the particle size selected, between, for example, 0.2 and 500 μm, of a filtration which can, for example, be under vacuum, or even of an ultrafiltration, or of high performance chromatography, or any other technique chosen so as not to detrimentally modify the extracts.

Preferably, the step of recovering the organic and/or organomineral molecules can correspond to a concentration of the molecules by elimination of all or part of the liquid. This step can comprise, for example, precipitation with a counter-solvent, evaporation at low pressure, sublimation after freezing (lyophilization), without this list being limited.

The counter-solvent may be, for example, a solvent as described above.

In the present invention, the evaporation or the sublimation at low pressure can be carried out, for example, at a pressure included in the range of from 0.1 to 5000 Pa (50 to 0.001 hPa), preferably between 0.1 and 4000 Pa, more preferably between 0.1 and 25 Pa, even more preferably between 0.1 and 20 Pa.

A subject of the present invention is also the organic and/or organomineral molecules that can be obtained by means of the method of the invention.

A subject of the present invention is also the use of the organic and/or organomineral molecules that can be obtained by means of the method of the invention, for producing a cosmetic composition.

A subject of the present invention is also a method for producing a cosmetic composition, which comprises implementing the method for extracting organic and/or organomineral molecules of the present invention and mixing the organic and/or organomineral molecules obtained in a cosmetically acceptable support.

A subject of the present invention is also a cosmetic composition comprising at least one organic and/or organomineral molecule obtained by means of the method of the invention and a cosmetically acceptable support.

In the present invention, the term “cosmetic composition” is intended to mean any composition for cosmetic purposes, i.e. a composition that can be brought into contact with the superficial parts of the human body, for example the epidermis, the body-hair and head-hair systems, the external organs, the teeth and the mucous membranes.

It may be, for example, a liquid composition, an emulsion, an ointment, a foam, a paste or a gel.

In the present invention, the term “cosmetically acceptable support” is intended to mean any compound known to those skilled in the art which makes it possible to produce a cosmetic composition.

The cosmetic composition according to the invention may also comprise one or more cosmetic adjuvants chosen from conditioning agents of ester type; antifoams; moisturizers; emollients; plasticizers; inorganic thickeners, organic thickeners, which may be polymeric or nonpolymeric and associative or nonassociative; water-soluble and liposoluble, silicone or nonsilicone sunscreens; permanent or temporary dyes; fragrances; preservatives; ceramides and pseudoceramides; vitamins and provitamins; proteins; sequestering agents; solubilizing agents; basifying agents; anti-corrosion agents; reducing agents or antioxidants; oxidizing agents; and inorganic fillers.

In the present invention, the cosmetic composition may be, for example, an anti-aging composition, for example a cosmetic composition which stimulates densification of the papillary dermis and which stimulates the synthesis of dermal-epidermal junction proteins.

A subject of the present invention is also a method for producing a pharmaceutical composition, which comprises implementing the method for extracting organic and/or organomineral molecules of the present invention and mixing the organic and/or organomineral molecules obtained in a pharmaceutically acceptable support.

According to the invention, pharmaceutically acceptable may be, for example, a medicament intended for increasing bone, cartilage or skin tissue regeneration and/or a medicament intended for the treatment of arthrosis, arthritis, osteoporosis or degenerative aging.

A subject of the present invention is also the use of the organic and/or organomineral molecules that can be obtained by means of the method of the invention, for producing a pharmaceutical composition and/or a medicament.

A subject of the present invention is also a pharmaceutical composition comprising at least one organic and/or organomineral molecule obtained by means of the method of the invention and a pharmaceutically acceptable support.

In the present invention, the term “pharmaceutical composition” is intended to mean a liquid composition, an emulsion, an ointment, a foam, a paste, a scored tablet, a gel capsule, an effervescent tablet, a dermal patch or an injectable device, without the form being limiting.

In the present invention, the term “pharmaceutically acceptable support” is intended to mean an inert diluent, such as sorbitol, sugar, mannitol, microcrystalline cellulose, starch, sodium chloride, sodium phosphate, calcium carbonate, calcium phosphate, calcium sulfate or lactose, for example lactose monohydrate.

In the present invention, the medicament can be used, for example, in the treatment or prevention of diseases related to a decrease in bone density and mass.

In the present invention, the medicament may be used, for example, in the treatment or prevention of diseases chosen from the group comprising osteopenia, including genetic osteopenia, osteoporosis, degenerative aging, rheumatological infections, including arthrosis, arthritis, and all the diseases which affect the skeleton.

The medicament may also be used, for example, in the treatment of skin diseases, for example diseases with genetic, psychosomatic or pathological origins, or caused by intrinsic and extrinsic aging or by environmental attacks or stresses. It may, for example, be a disease related to ultraviolet (UV) solar radiation, to infrared (IR), to an excessive humid atmosphere, to conditioned air and/or to dust.

A subject of the present invention is also the use of the organic and/or organomineral molecules that can be obtained by means of the method of the invention, for producing a medicament intended for increasing bone, cartilage or skin tissue regeneration.

In the present invention, the expression “increasing bone, cartilage or skin tissue regeneration” is intended to mean, for example, the reformation of tissues by stimulation of cell activity inducing, for example, osteogenesis, restructuring of the epidermis, redensification of the dermis via the production of extracellular matrix, or of the activity of the epidermal junction, regulation of the activity of the hypodermis, inter alia. According to the invention, the increase in bone, cartilage or skin tissue regeneration can be carried out in vivo and/or in vitro or for the culture of tissues, cells or cell assemblies.

In the present invention, the medicament can be administered, for example, topically, esophageally, subcutaneously or intravenously or by any other suitable administration method. The medicament may be formulated in any way known to those skilled in the art, according to the administration.

A subject of the present invention is also the use of organic and/or organomineral molecules that can be obtained by means of the method of the invention, for treating diseases chosen from the group comprising osteopenia, osteoporosis, degenerative aging, rheumatological infections, including arthrosis, arthritis, orthopedic diseases and dermatological diseases.

A subject of the present invention is also the use of organic and/or organomineral molecules that can be obtained by means of the method of the invention, for application with an implant or on an implant for, for example, increasing the regeneration of tissues, for example bone, cartilage or skin tissues, in all types of bone and other tissues; for example, in the vertebral column, the limbs (compact bone, spongy bone), the flat bones of the face and of the cranium, the thoracic skeleton or the like.

A subject of the present invention is also the use of organic and/or organomineral molecules that can be obtained by means of the method of the invention, for producing bone grafts by stimulation of osteoblasts or fibroblasts (osteoinduction).

Other advantages may also emerge to those skilled in the art on reading the examples below, illustrated by the appended figures, given by way of illustration.

### BRIEF DESCRIPTION OF THE FIGURES

FIG. 1 represents cell culture wells. The cells are cultured in the presence or in the absence of extract (T) of limestone rocks, obtained by means of a prior art method (IM, NAC, AGH) or one of the embodiments of the method of the invention (AGA, AGB).

FIG. 2 represents histological sections of cells after 5 days of culture.

FIG. 2A represents a section of an untreated control explant maintained under survival conditions.

FIG. 2B represents a section of an explant cultured under conditions identical to those of the control having received a treatment with extract of limestone rock.

FIG. 3A represents a histological section of an untreated explant of human skin maintained in a state of survival for 9 days, and constitutes a control for the section of FIG. 3B.

FIG. 3B represents a histological section of a same explant of human skin maintained in a state of survival under the same conditions as FIG. 3A. treated for 9 days with the Orgon organomineral extract obtained by means of the method described in example 1, showing the stimulation of the neutral GAGs and acidic GAGs (hyaluronic acid).

FIG. 4A represents a control histological section of an untreated explant of skin maintained in a state of survival for 9 days, so as to constitute a control for the histological section of FIG. 4B.

FIG. 4B represents a histological section of a same explant of human skin maintained in a state of survival under the same conditions as for FIG. 4A, treated for 9 days with the Orgon organomineral extract, and shows the stimulation of the neutral GAGs and acidic GAGs (hyaluronic acid).

### EXAMPLES

### Example 1

### Extraction of Organic and/or Organomineral Molecules

A calcium carbonate rock, 50 g of rock from Orgon, was mixed, in a 1000 ml Nalgene stoppered container, with a solution comprising 238 ml of GPR RECTAPUR absolute ethanol (organic solvent) from the company VWR International SAS, 12 ml of milliQ water obtained with a complete water purification system from the company Thermo Scientific Barnstead (Diamond RO osmosis system and Easypure II polishing system) and 1 ml of inorganic acid (37% or 12N HCl) from the company Carlo Erba. The pH of the resulting solution was measured with a pH-meter from the company Hach-Lange (Titralab 854) and the pH was 1.58.

This example was carried out at a temperature of 23° C.

The mixing was carried out with a Heidolph rotary eluting device at a rotation speed of 10 rpm.

In this experiment, three identical mixtures were prepared. Various extraction times were tested.

The extraction of the molecules was obtained after increasing times by centrifugation at 3000 g (Jouan CR 3.12 centrifuge, France) for 20 minutes, refrigerated at 4° C. The supernatant was then filtered, using a filter holder for membrane vacuum filtration (Whatman) with a Millipore filter: Durapore hydrophilic polyvinylidene fluoride (PVDF) membrane with a 0.22 μm cut-off. The solutes were then evaporated off under vacuum using an evaporator (Büchi Rotovapor R-200) so as to remove the organic solvent. Finally, they were freeze-dried using a Christ LyoDisplay Alpha 2-4 freeze-dryer equipped with a trap at −85° C. A powder was obtained.

The molecules extracted were analyzed by plasma emission spectrometry (ICP-OES, Ultima Jobin Yvon), by analytical high performance liquid chromatography (HPLC) (Thermo, BioBasic SEC-300 column and UV 2000 detector), PyroGC/MS (THERMOfinnigan) and by SDS-PAGE electrophoresis (Mini ProteanTetra Cell, Biorad).

Table 1 below summarizes the characteristics of the extracts obtained.

TABLE 1
Characteristics of the mixtures prepared, amounts extracted and extraction
yield

| | | | Yield by |
|---|---|---|---|
| | Time | Extract | weight |
| Samples | (hours) | in mg | (%) |
| 50 g Orgon + 238 ml absolute | 15 h | 572.3 | 1.14 |
| ethanol + 12 ml milliQ H₂O + 1 ml | | | |
| 37% HCl | | | |
| 50 g Orgon + 238 ml absolute | 24 h | 678.6 | 1.36 |
| ethanol + 12 ml milliQ H₂O + 1 ml | | | |
| 37% HCl | | | |
| 50 g Orgon + 238 ml absolute | 48 h | 646.7 | 1.29 |
| ethanol + 12 ml milliQ H₂O + 1 ml | | | |
| 37% HCl | | | |

As demonstrated in this example and in the table above, the extraction yield obtained with an example of implementation of the method of the invention was constant at approximately, on average, 1.3% of extract.

### Example 2

### Extraction with a Prior Art Method

The protocol described in European patent EP 0869805 was applied to 8 samples of rock from Orgon with two different dilutions and increasing extraction times. The devices and products used in this example are identical to those used in patent EP 0869805.

Various extraction times were tested.

Results: Table 2 below summarizes the conditions tested.

TABLE 2
Results of experiments for extraction of organic molecules with the
method of patent EP 0869805

| | Time | Extract |
|---|---|---|
| Sample | (hours) | weight (mg) |
| 50 g Orgon + 250 ml milliQ H₂O | 15 h 30 | 2.8 |
| 50 g Orgon + 250 ml milliQ H₂O | 19 h 30 | 3.3 |
| 50 g Organ + 250 ml milliQ H₂O | 24 h | 1.5 |
| 50 g Orgon + 250 ml milliQ H₂O | 48 h | 2 |
| 50 g Orgon + 500 ml milliQ H₂O | 15 h 30 | 2.6 |
| 50 g Orgon + 500 ml milliQ H₂O | 19 h 30 | 2.2 |
| 50 g Orgon + 500 ml milliQ H₂O | 24 h | 2.8 |
| 50 g Orgon + 500 ml milliQ H₂O | 48 h | 2.6 |

The amounts of extract with this method were about a milligram. The extraction yield by weight with the prior art method is about 0.0056%.

As demonstrated in example 1, the method of the invention makes it possible to obtain a yield of 1.3%, i.e. at least several hundred times higher (approximately 232) than the prior art method.

### Example 3

### Extraction of Organic and/or Organomineral Molecules According to Various Extraction Times

The compounds of this example are identical to those of example 1. In this example, the proportion of acid was increased compared with example 1 and various extraction times were tested. 50 g of rock from Orgon were mixed with a solution comprising 238 ml of absolute ethanol (organic solvent), 12 ml of absolute water and 2 ml of inorganic acid (HCl at 37%). The pH of the solution was 1.2.

Various extraction times were tested in this example. The pH of the solution was lower than that of the solution of example 1.

Table 3 below summarizes the conditions and the amount of organic and/or organomineral molecules obtained.

TABLE 3
Extraction results

| | | | Yield by |
|---|---|---|---|
| | Time | Extract | weight |
| Sample | (hours) | weight (mg) | (%) |
| 50 g Orgon + 238 ml absolute | 15 h | 1219.6 | 2.44 |
| ethanol + 12 ml milliQ H₂O + | | | |
| 2 ml 37% HCl | | | |
| 50 g Orgon + 238 ml absolute | 24 h | 1643.6 | 3.29 |
| ethanol + 12 ml milliQ H₂O + | | | |
| 2 ml 37% HCl | | | |
| 50 g Orgon + 238 ml absolute | 48 h | 1574.7 | 3.15 |
| ethanol + 12 ml H₂O milliQ + | | | |
| 2 ml 37% HCl | | | |

In this experiment, it was shown that the decrease in pH made it possible to increase the extraction yield, which was then between 2.4% and 3.3%.

As demonstrated in this example, a decrease in the pH of the mixture made it possible to increase the extraction yield. In this example, the amounts extracted are 600 to 800 times higher than the amounts extracted with the prior art method presented in example 2.

This example therefore clearly demonstrates that the method of the invention makes it possible to very greatly increase the amounts of extracts of limestone rocks and thus makes it possible to optimize the method and to reduce the costs.

### Example 4

### Example of Implementation of the Method of the Invention with Larger Volumes of Aqueous and Organic Solvents

The reactants and implementation conditions of this example are identical to those of example 1.

In this example, 50 g of rock from Orgon were mixed with a solution comprising 476 ml of absolute ethanol (organic solvent), 24 ml of absolute water and 2 ml of inorganic acid (HCl at 37%). The pH of the solution was 1.51.

The results and the extraction time are indicated in table 4 below.

TABLE 4
Extraction results

| | | | Yield by |
|---|---|---|---|
| | | Extract | weight |
| Protocol | Time (hours) | weight (mg) | (%) |
| 50 g OrgonT + 476 ml | 15 h | 1601.5 | 3.20 |
| absolute ethanol + 24 ml | | | |
| milliQ H₂O + 2 ml 37% HCl | | | |
| 50 g OrgonT + 476 ml | 24 h | 1251.1 | 2.50 |
| absolute ethanol + 24 ml | | | |
| milliQ H₂O + 2 ml 37% HCl | | | |

As demonstrated in this example, the rock/solvent proportion in the method does not change the yield at a constant amount of acid.

### Example 5

### Example of Implementation of the Extraction Method According to the pH

The compounds and the implementation conditions are identical to those of the previous examples. 50 g of rock from Orgon were mixed with a solution comprising 250 ml of absolute ethanol; the aqueous fraction was minimized by using only the inorganic acid, plus 1 ml and then 2 ml with a pH approximately equal to 0.

Various times were tested. As presented in table 5, the yield 2O increases with the amount of acid only: 1.2% then 3.2%.

Moreover, various amounts of solvents were tested.

Table 5 below summarizes the conditions and the results obtained.

TABLE 5
Results of extraction according to various amounts of solvent.

| | | Extract | Yield by |
|---|---|---|---|
| | Time | weight | weight |
| Protocol | (hours) | (mg) | (%) |
| 50 g Orgon + 250 ml of absolute | 15 h | 596.6 | 1.19 |
| ethanol + 1 ml 37% HCl | | | |
| 50 g Orgon + 250 ml of absolute | 24 h | 525.1 | 1.05 |
| ethanol + 1 ml 37% HCl | | | |
| 50 g Orgon + 250 ml of absolute | 48 h | 664.7 | 1.33 |
| ethanol + 1 ml 37% HCl | | | |
| 50 g Orgon + 250 ml of absolute | 15 h | 1548.6 | 3.10 |
| ethanol + 2 ml 37% HCl | | | |
| 50 g Orgon + 250 ml absolute | 24 h | 1646.9 | 3.29 |
| ethanol + 2 ml 37% HCl | | | |
| 50 g Orgon + 250 ml absolute | 48 h | 1564.2 | 3.13 |
| ethanol + 2 ml 37% HCl | | | |
| 50 g OrgonT + 500 ml absolute | 15 h | 1315.4 | 2.63 |
| ethanol + 2 ml 37% HCl | | | |
| 50 g OrgonT + 500 ml absolute | 24 h | 1670 | 3.34 |
| ethanol + 2 ml 37% HCl | | | |

As presented in table 5, if the amount of organic solvent is increased, the yield remains unchanged at a constant amount of acid, for example for 500 ml of organic solvent, the extraction yield remains equal to approximately 3%.

### Example 6

### Extraction using Varying Particle Sizes

The implementation conditions are identical to those in example 1.

The samples of rock from Orgon were crushed beforehand by the supplier (Omya SAS). A screening column was used to characterize the product as received from the supplier. The screening column comprises the various Stages: 500 μm, 200 μm, 100 μm and 50 μm (supplied by OST Inox). Table 6 gives the percentage of each fraction. A series of samples was crushed from blocks of rocks. The blocks were crushed using a laboratory mill Oka-Werke A11 basic). The crushed material was then screened in the same screening column. 50 g of each of the fractions were produced, with the crushing being increasingly fine.

The crushed materials were then mixed with a solution comprising 238 ml of absolute ethanol, 12 ml of absolute water and 12 ml of inorganic acid (HCl at 37%). Table 6 below summarizes the amounts of extracts obtained according to the size of the crushed material.

TABLE 6
Characterization of the particle size of the Orgon powder

| | Amount measured | Percentage by |
|---|---|---|
| Particle size | (g) | weight (%) |
| f > 500 μm | 0.0036 | 0.01 |
| 200 μm < f < 500 μm | 0.4205 | 0.85 |
| 100 μm < f < 200 μm | 6.6440 | 13.42 |
| 50 μm < f < 100 μm | 11.7227 | 23.67 |
| f < 50 μm | 30.7354 | 62.06 |

The amount of extracts that is obtained with the various particle sizes is presented in table 7 below.

TABLE 7
Yield from extraction of the limestone rock from Organ according to
particle size

| | | | Yield by |
|---|---|---|---|
| | Time | Extracts | weight |
| Particle size | (hours) | (mg) | (%) |
| 50 g: 500 μm < f < 1000 μm | 24 h | 456.2 | 0.9 |
| 50 g: 200 μm < f < 500 μm | 24 h | 543.6 | 1.09 |
| 50 g: 100 μm < f < 200 μm | 24 h | 1343.6 | 2.69 |
| 50 g: 50 μm < f < 100 μm | 24 h | 1619.6 | 3.24 |
| 50 g: f < 50 μm (standard powder) | 24 h | 1643.6 | 3.29 |

As demonstrated in this example, the extraction exists irrespective of the particle size. More particularly, the yield increases when the particle size decreases, and remains constant when the particle size is less than 200 μm.

### Example 7

### Study of the Effect of the Extracts on Bone Mineralization

The various steps of bone differentiation CaO be studied in vitro on cell cultures. The ability of the extracts to stimulate bone mineralization mechanisms was tested according to the protocol described in Bellows et al., 1991, 1992 (Refs 8, 9).

In this example, the extracts of rocks were obtained from the limestone of the Orgon deposit, according to the three extraction conditions described in table 8.

The extraction was carried out for 15 hours according to the protocol previously described. The solvent was then evaporated off and the pellet was freeze-dried.

TABLE 8
Conditions for obtaining the extracts and yield from the methods used

| | | | | Yield by |
|---|---|---|---|---|
| | | | | weight |
| Conditions | Solvent | Water | Acid | (%) |
| AGA | 475 ml absolute | 25 ml | 1.35 ml 37% | 0.905 |
| | ethanol | | HCl | |
| AGB | 475 ml absolute | 25 ml | pH = 3.7 | 0.014 |
| | ethanol | | | |
| AGH | — | 500 ml | — | 0.0035 |
| | | milliQ H₂O | | |

—: not present in the solution

The extract obtained according to the method of the invention corresponds to the AGA condition. The AGB condition is obtained according to the method, but without being optimized.

The AGH condition corresponds to the extract obtained according to the protocol of the abovementioned European patent.

After having freeze-dried the 3 extracts, their activity was tested using in vitro cell cultures of mouse MC3T3-E1 cells (ATCC, USA). This is an osteoblast line of predifferentiated cells, committed to the osteoblast pathway, capable of mineralizing after having reached a maturation stage.

The MC3T3 cells were cultured for 25 days at 37° C. under 5% CO₂ in an alpha Minimum Essential medium (α-MEM medium) (alpha-modification, Sigma M4526) containing 50 U/ml of penicillin and 50 μg/ml of streptomycin (Gibco-Invitrogen), 10% of fetal calf serum (HyClone-Perbio) and 2 mM L-glutamine (GibcoBRL). The AGA, AGB and AGH extracts were added to this culture medium, namely 200 μg of dry extract per ml of nutritive medium. These tests were compared with the control without addition (T), and also with a known nutritive medium with ascorbic acid and beta-glycerophosphate (IM).

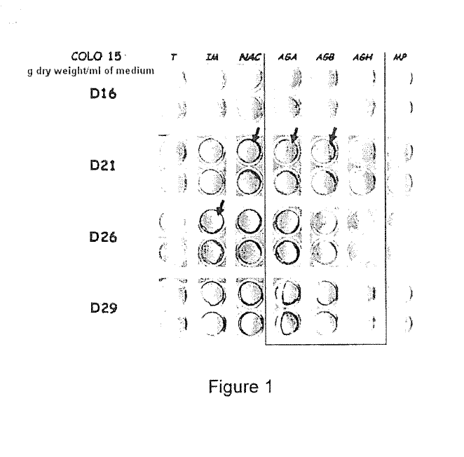

The results of this experiment, FIG. 1, showed a very clear stimulation of the development of the cells when the nutritive medium comprises extracts of limestone rocks according to the AGA and AGB conditions.

The rock extracts obtained with the AGA and AGB conditions stimulate mineralization from 21 days onward, as for the extracts of fresh nacre (NAC). The reference nutritive medium reached the same stage after 26 to 29 days. On the other hand, the AGH rock extract obtained with the prior art protocol did not stimulate mineralization. The extract of nacre (NAC) obtained according to the prior art method allows stimulation of mineralization as expected and confirms the reactivity of the cells.

As demonstrated in this example, when the preosteoblast cells are exposed to the extracts obtained by means of the method of the invention, they trigger the bone mineralization mechanism from the 3rd week onward, whereas the specific inducers produce the same effect only at the end of the 4th week of culture.

The limestone material extracts obtained according to the method of the invention therefore make it possible to stimulate mineralization, unlike the limestone material extract obtained by means of the method of patent EP 0869805 of the prior art. The extracts of rock according to the method of the invention therefore clearly have an activity on biological tissues.

### Example 8

### Effect of the Extracts of Rocks on Explants of Human Skin

In this example, the cosmetic applications were demonstrated on explants of human skin maintained under survival conditions. The explants originated from an abdominaplasty on a 38-year-old woman. 21 explants of whole skin were placed in a state of survival in BEM medium according to the method of Bio-EC, France.

3 groups of 6 explants and 1 group of 3 explants were formed as presented below:

| | | |
|---|---|---|
| Control | T0 | 3 explants |
| untreated batch | T | 6 explants |
| CMC gel | E | 6 explants |
| product P1 | P1 | 6 explants |

The control group T0 corresponded to the 3 explants that were removed at D0 (day zero).

The untreated group T corresponded to the group of 6 explants that were maintained under survival conditions using the BEM culture medium.

The group E, which was CMC (carboxymethylcellulose) gel, corresponded to the group of 6 explants that were maintained under survival conditions and to which the excipient, under namely the CMC gel, was applied without active agents.

The product P1 group corresponded to the group of 6 explants maintained under survival conditions and to which the CMC gel containing the mixture of extract of rock according to the invention, concentrated at 2%, was applied.

The treatment was carried out by topical application of 2 mg of test product per explant. This treatment was carried out every day. The culture media were renewed 50/50 on D2, D5 and D7, i.e. 2 days after, 5 days after and 7 days after the beginning of the experiment.

On D0, the 3 explants of the TO group were removed and cut in half. One part was fixed with buffered formol for observation of the general morphology. The other was frozen and stored at −80° C. On D5 and on D9, 3 explants of each group were removed and treated in the same way.

Histological preparations were prepared according to the protocols conventionally used: paraffin sections after staining with Masson's trichrome, Goldner variant according to the MO-H-157 procedure. The observations of general morphology presented herein were carried out by optical microscopy, using a Leica DMLB microscope, with the x 40 objective. The images were taken with a Sony DXC 390P tri-CCD camera and stored using the Leica IM1000 data archiving software.

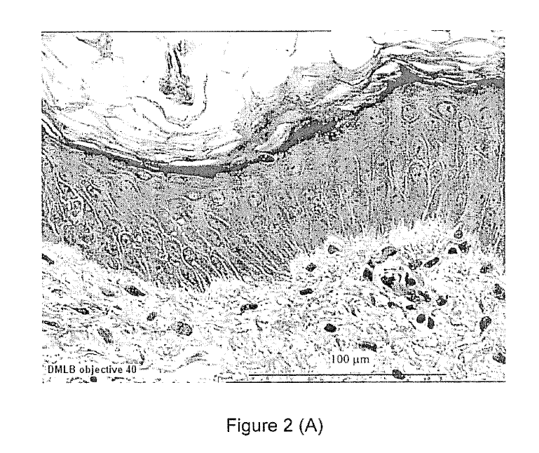

Compared with the untreated control explants, after 5 days, the explants on application of the rock extracts show that the stratum corneum is moderately thick and laminated, slightly keratinized at the surface and very clearly keratinized at its base (FIG. 2B). There is slight parakeratosis. The epidermis shows 6 to 7 cell strata with good morphology. The control sections (FIG. 2A), like the starting group, show only 4 to 5. The outline of the dermal-epidermal junction is very sharp. The papillary dermis shows quite thick collagen fibers forming a dense network. It is well cellularized.

After 9 days of treatment, a clear increase in the thickness of the stratum corneum was noted (sign of considerable cell renewal and of quite good terminal differentiation) with an increase in epidermal thickness (FIG. 2B). The collagen network, in the papillary dermis, was also more dense.

As presented in this example, a study of the general morphology of explants of human skin was carried out under nonoptimized standard conditions. It showed a very clear epidermal and dermal stimulating activity with the rock extract from 5 days of application onward, which was reinforced after 9 days (not illustrated): better anchoring with restructuring of epidermal cell stratification, restoration of the outline of the dermal-epidermal junction and also a denser collagen network in the papillary dermis.

This example therefore clearly shows that the extract obtained by means of the present invention makes it possible, in addition to stimulating the formation, mineralization and regulation of bone mass, to obtain a restructuring and regulating effect on the whole of the skin activity.

### Example 9

### Example of Limestone Rock Characteristics

The limestone rocks that can be used in the method of the invention can be chosen, for example, on the basis of their total organic carbon (TOC) content, even though the technique is only an indication and generally not very sensitive, and only the extraction according to the patent counts.

A pyrolytic method known as Rock-Eval pyrolysis was used in this example, diverted from its customary use, namely mineral oils and contaminations. The table below shows that it is possible to detect a peak related to the cracking, under a neutral atmosphere, of organic constituents, by means of an FID (flame ionization) detector (Ref 8) (mg of hydrocarbons formed via cracking as a function of temperature and via the temperature of its maximum). This peak, even very low, on the limestone rocks selected from a certain number of producers, does not appear on the precipitated calcium carbonate (PCC) sold by VWR International.

TABLE 9
Examples of limestone rock characteristics

| | | | Rock-Eval |
|---|---|---|---|
| | | | Existence of |
| | | | the peak |
| | | XRD | (temperature |
| | Exploiting | crystallographic | of maximum |
| Limestone rocks | company | composition | in ° C.) |
| Orgon | Omya SA | Calcite | Yes |
| (Bouches-du- | France | | (Tmax = 455) |
| Rhône, France) | | | |
| PCC (precipitated | VWR | >99.5% | No |
| calcium carbonate) | International | pure calcite | |
| | SA | | |
| Saint Germain | Solvay SA | Calcite | Yes |
| (Meuse, France) | | | (Tmax = 463) |
| Héming Block LF | Holcim | Calcite (quartz, | Yes |
| (Moselle, France) | France | dolomite) | (Tmax = 429) |
| Euville | Rocamat | Calcite | Yes |
| (Meuse, France) | | | (Tmax = 445) |
| Saint Maximin | Rocamat | Calcite | Yes |
| (Chantilly, Oise, | | | (Tmax = 446) |
| France) | | | |
| Sainte Croix de | IMERYS | Calcite | — |
| Mareuil (Dordogne, | | | |
| France) | | | |

—: not tested

As demonstrated in the table above, the various rocks tested all show a pyrolysis peak comparable to that of the Orgon rock.

These rocks are calcium carbonates (calcite) which can be used in the method of the invention.

### Example 9

### Cosmetic Composition

A cosmetic dermal re-densifying cream is prepared from the extracts obtained in example 1. It is a stable oil-in-water emulsion in which the oily phase is dispersed in an aqueous phase with an emulsifier based on a fatty acid ester: glucose palmitate and sucrose palmitostearate (5%). The oily phase is prepared with sunflower oil (13%) and jojoba oil (5%). The aqueous phase contains bacteriostatic substances. A concentration of 2% of active agent was used and added to the aqueous phase.

The preparation obtained is tested by topical application to skin explants under the conditions described in example 8. The sections of explants at 9 days show, with the excipient alone, an epidermal structure in good survival condition comparable to the morphology of the control explants at the beginning of the experiment (DO). Application of the cosmetic cream induces a clear increase in epidermal thickness (7/8 cell strata) and, in the papillary dermis, a more dense collagen network. The structure of the dermal-epidermal junction is clearly remodeled with pronounced anchoring in the dermis.

### Example 10

### Pharmaceutical Composition

A pharmaceutical composition for renewal and restructuring of the hydrolipidic barrier of the skin was prepared for a topical application to the skin, for carrying out a natural peel without any physical abrasion and therefore without weakening of the epidermal protection. The excipient used is a conventional base described in the table below.

TABLE 10
Pharmaceceutical composition base

| Benzyl alcohol | 2.200 | g |
|---|---|---|
| Isopropyl palmitate | 2.200 | g |
| Glycerol | 4.000 | g |
| Sorbitol 70 percent | 5.000 | g |
| Emulsifiable wax | 12.500 | g |
| Lactic acid and | q.s. pH = 4.8 to 5.2 | |
| sodium hydroxide | | |
| Purified water | q.s. 100.00 | g |

The active agents were added in a proportion of 2% on the basis of the extracts obtained during example 3.

The pharmaceutical composition obtained is tested by topical application to skin explants under the conditions described in example 8. After 9 days, a verification was carried out in order to verify that the explants treated with the excipient alone retain an epidermal structure in good survival condition comparable to the morphology of the control explants at the beginning of the experiment (DO). The sections of explants treated with the pharmaceutical composition show, after 9 days, considerable cell renewal and good terminal differentiation, with a moderate increase in epidermal thickness (6/7 cell strata). In 9 days, a peel is observed, followed by renewal of the hydrolipidic barrier of the skin.

### Example 10

### Comparative Example: Extraction with a Prior Art Method Described in Document FR 2 919 186

The protocol described in document FR 2 919 186 was applied to a sample of rock from Orgon and a sample of Falun [shelly deposits] of Touraine as described in document FR 2 919 186.

The devices and products used in this example are identical to those used in document FR 2 919 186. The two materials were crushed so as to obtain an average particle size of less than 1 μm. Each of the powders was placed in demineralized water containing a mixture of preservatives: potassium sorbate at 0.3% by weight, sodium benzoate at 0.4% by weight, sodium dehydroacetate at 0.1% by weight and benzyl alcohol at 0.6% by weight, in a tank fitted with a stirrer, and maintained at ambient temperature, i.e. 22° C., in a proportion of 20% by weight, as indicated in document FR 2 919 186. The pH of the solution was verified in order to confirm that said pH was indeed neutral, as described in the abovementioned document, i.e. pH=7.1. Stirring was maintained for 48 hours at a temperature of 22° C. as indicated in document FR 2 919 186. The mixture was then filtered in order to separate the residual mineral suspension from the liquid.

An analysis of the minerals in solution was then carried out on the liquid by ICP/AES (Jobin Yvon Ultima 2 (registered trademark)) in order to evaluate the extraction yield with the method of document FR 2 919 186. In the sample of falun [shelly deposits], the inventors measured a value of 205 mg of Ca per liter and 23.5 mg of Si per liter of solution (1396 mg of residue after freeze-drying). They found a value of 245 mg of Ca for the rock from Orgon and an undetectable Si value. The yield by weight relative to the weight from Orgon rock is 0.0012% using this prior art method described in document FR 2 919 186. The yield by weight obtained for the falun [shelly deposits] of Touraine using the same prior art method is 0.007%.

Thus, as demonstrated in this example, the extraction yield of the method of the invention makes it possible to obtain a yield that is more than 1000 times higher than the methods described in the prior art.

### Example 11

### Cosmetic Activities of the Extracts Obtained using the Method of the Invention, According to the Provenance of the Limestone Material

This example is carried out under the same conditions as example 8 above.

An abdominoplasty from a 53-year-old woman was used. Several batches of explants were obtained. In this example, all the extracts were applied according to the protocol described in example 8 above: topical application, every day, of 2 mg of CMC gel containing the rock extract according to the invention, concentrated at 1%.

The extract of Orgon rock as obtained in example 1 was used to test a batch of explants denoted CA2.

An extract of Saint Germain rock as obtained according to the same method was used to test a batch of explants denoted CB6.

After treatment for 5, 9 or 10 days on the explants, histological sections were prepared by fixing the explants for 24 hours in buffered formol, dehydrating and impregnating with paraffin using an automated processor for dehydration (Leica 1020) and embedding in blocks (embedding station (Leica EG 1160)). 5 μm sections were then cut on a microtome (Minot, Leica RM2125) and attached to superfrost (registered trademark) silanized histological glass slides. The stainings and the immunochemical labelings were carried out on the sections under the following conditions:
The glycosaminoglycans (GAGs) are visualized by staining with alcian blue—P.A.S. on the sections previously described (also known to those skilled in the art as Mowry staining).
Collagen I was labeled on frozen sections with a rabbit anti-collagen I polyclonal antibody (Monosan, ref: PS 047), at 1/500th for 1 h at ambient temperature, with a biotin/streptavidin amplifying system, revealed by FITC. The nuclei were counterstained with propidium iodide.
Collagen III was labeled on frozen sections with a goat anti-collagen III polyclonal antibody (SBA ref: 1330-01), at 1/320th for 2 h at ambient temperature, with a biotin/streptavidin amplifying system, revealed by DAB. The nuclei were counterstained with Masson's hemalun.
Collagen IV was labeled on frozen sections with a goat anti-collagen IV polyclonal antibody (SBA ref: 1340-01), at 1/60th for 2 h at ambient temperature, with a biotin/streptavidin amplifying system, revealed by fluorescein isothiocyanate (FITC). The nuclei were counterstained with propidium iodide.
Laminin-5 was labeled on frozen sections with a goat anti-laminin-5 monoclonal antibody, clone P3E4 (SBA ref: 1340-01), at 1/320th for 1 h 30 at ambient temperature, with a biotin/streptavidin amplifying system, revealed by FITC. The nuclei were counterstained with propidium iodide.

The results are evaluated by comparison with controls carried out on batches of explants treated with the excipient only.

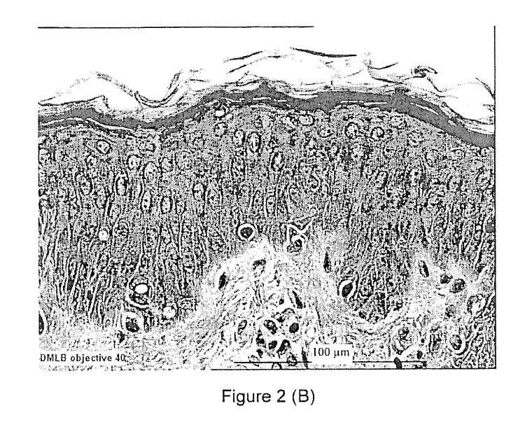

The CA2 batch treated for 10 days with the Orgon organomineral active agent obtained in example 1 unexpectedly showed a clear overexpression of acidic GAGs (GAGsA) in the papillary dermis, as represented in FIG. 3 B. These GAGs are known to be essentially hyaluronic acids, a moisturizing factor highly sought after in the dermopharmaceutical and cosmetics industries.

A moderate overexpression of neutral GAGs (GAGsN) (growth factor store) and of collagen type I (Coll I) and III (Coll III) (juvenile collagen) was also observed. These effects can advantageously produce a stimulation of densification of the papillary dermis, which is an anti-wrinkle effect sought after in the cosmetics industry (see table 11).

The CB6 batch treated for 10 days with the organomineral extract obtained from the limestone material of the Saint Germain quarry showed, compared with the control batch, a clear overexpression of collagen IV (Coll IV) (support tissue collagen), of laminin-5 (Lam 5) and of collagen type III or juvenile collagen (see table 11).

TABLE 11
Results obtained with the extracts obtained by means of the
method of the invention, compared with controls

| | | D9/D10 | | | | | |
|---|---|---|---|---|---|---|---|
| | Extract origin | | | Coll | Coll | Coll | |
| Batch | (extraction) | GAGsN | GAGsA | I | III | IV | Lam 5 |
| DA2 | Orgon (E) | [symbol] | [symbol] [symbol] [symbol] | [symbol] | [symbol] | [symbol] | [symbol] |
| DB6 | Euville (E) | [symbol] [symbol] | [symbol] | [symbol] | [symbol] | [symbol] [symbol] [symbol] | [symbol] [symbol] [symbol] [symbol] |
| DB2 | Nacre (E) | [symbol] | [symbol] | [symbol] [symbol] | [symbol] | [symbol] | [symbol] |

Decrease:
slight: [symbol] ;
moderate: [symbol] [symbol] ;
quite clear: [symbol] [symbol] [symbol] ;
clear: [symbol] [symbol] [symbol] [symbol] .
Increase:
slight: [symbol] ;
moderate: [symbol] [symbol] ;
quite clear: [symbol] [symbol] [symbol] ;
clear: [symbol] [symbol] [symbol] [symbol] or
unchanged: [symbol] .

As demonstrated in this example, the extracts obtained by means of the method of the present invention therefore have an effect, for example, on the proteins involved in the dermis and the dermal-epidermal junction by stimulating their activities.

Thus, this example clearly demonstrates that the extracts obtained by the means of the method of the invention can be used, for example, in the cosmetics field, for example for an anti-aging effect by restructuring of the outline of the dermal-epidermal junction and stimulation of its activity.

This example demonstrates that, under nonoptimized conditions, extracts as obtained by means of the method, from various limestone materials, have an activity targeted at various dermal-epidermal levels.

### Example 12

### Dermatological Activities of the Extracts Obtained by Means of the Method with Various Limestone Materials

The activity tests of this example are carried out on explants of human skin maintained in a state of survival under the same conditions as those of example 11 above.

Stainings and labelings are obtained as described in example 11 above on GAGs, on collagens type I, III and IV, and on laminin-5. Other activities are evaluated as follows.

Cells in mitosis were labeled with a monoclonal antibody, anti-Ki67 (clone 7B11 from Zymed, mouse IgG1), with a biotin/streptavidin amplifying system (Vectastain RTU Universal Vector) and revelation by VIP.

G6PDH was labeled on Bouin-fixed paraffin sections with a mouse anti-G6PDH polyclonal antibody (Rockland ref: 200-1153), at 1/150th for 24 h at ambient temperature (20° C.), with a biotin/streptavidin amplifying system revealed by fluorescein isothiocyanate (FITC). The nuclei were counterstained with Masson's hemalun.

Loricrin was labeled on frozen sections with a rabbit anti-loricrin polyclonal antibody (Covance ref PRB-145), at 1/4000th overnight at 4° C., with a biotin/streptavidin amplifying system and revealed by FITC. The nuclei were counterstained with propidium iodide.

LEKTIs are serine protease inhibitors (lympho epithelial kazal type inhibitor). They were labeled using a mouse anti-LEKTI monoclonal antibody (Santa Cruz, ref sc-32330), at 1/800th overnight at 4° C., with a Vectastain RTU Universal VECTOR avidin/biotin amplifying system and revealed by FITC. The nuclei were counterstained with propidium iodide.

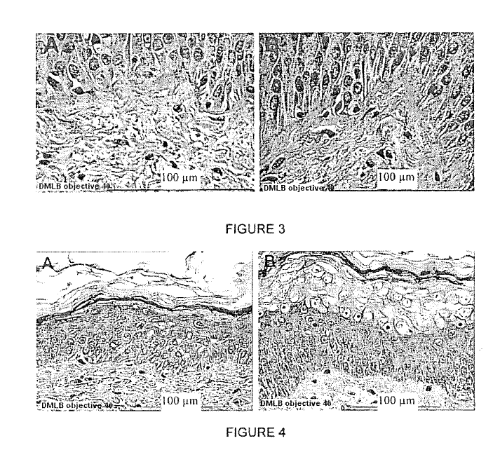

The DA2 batch treated for 9 days with the Orgon organomineral extract obtained in example 1 unexpectedly showed a regulation of complete turnover of epidermal stratification in 9 days with excellent morphology of the epidermis being retained, as represented in FIG. 4B. A new skin was therefore reconstituted in 9 days by simple topical application of the Orgon organomineral extract; the skin renews itself naturally three times more slowly, in 28 days.

The extracts obtained by means of the method of the invention can therefore be used in the case of skin burns, of stimulation of human explants undergoing growth, stimulation of tissues from synthesis in vitro, etc.

The DB6 batch was treated for 10 days with the organomineral active agent extracted from Euville stone obtained according to example 1. A stimulation of cell proliferation in the epidermis was observed with interest through overexpression of the Ki67 labeling of the cells in mitosis, as was a stimulation of LEKTIs in the granular layer (see table 12).

The DB2 batch was treated for 10 days with the extract obtained from Pinctada margaritifera nacre according to example 1. An overexpression of collagen type I was observed (see table 12).

TABLE 12
Results obtained with the extracts obtained by means of the method of
the invention, compared with controls

| | Extract origin | D5 | | | |
|---|---|---|---|---|---|
| Batch | (extraction) | Ki67 | G6PDH | Loricrin | Lekti |
| DA2 | Orgon (E) | [symbol] [symbol] | [symbol] | [symbol] | [symbol] |
| DB6 | Euville (E) | [symbol] [symbol] | [symbol] | [symbol] | [symbol] [symbol] |
| DB2 | Nacre (E) | [symbol] | [symbol] | [symbol] | [symbol] |

Decrease: slight: [symbol] ;
moderate: [symbol] [symbol] ;
quite clear: [symbol] [symbol] [symbol] ;
clear: [symbol] [symbol] [symbol] [symbol] .
Increase: slight: [symbol] ;
moderate: [symbol] [symbol] ;
quite clear: [symbol] [symbol] [symbol] ;
clear: [symbol] [symbol] [symbol] [symbol] or
unchanged: [symbol] .

This example demonstrates that the extracts obtained by means of the method of the invention, from various limestone materials of varied provenances, have specific stimulating effects on human skin. It shows that the age of the material may, in addition, produce beneficial effects: the limestone from Orgon (110 million years old) stimulates, in addition, the production of hyaluronic acid (GAGsA).

### Example 13

### Activity on the Skin of the Extracts Optimized by Fractionation

Various fractionated extracts were tested on explants of human skin ex-vivo, under the same conditions as examples 11 and 12 above.

In this example, the extract of Orgon rock was obtained by using the method in the following proportions: 50 g of the Orgon limestone that had been crushed are brought into contact with a solution of 500 ml of Milli-Q water, 10 ml of dichloromethane and 2 ml of hydrochloric acid.

The crude extract is used as it is to treat the explants of the batch denoted FA1 for 9 days. The treatment of the explants with the extract is carried out under the same conditions as those described in examples 11 and 12 above.

The same extract was demineralized and used for 10 days in order to test a batch of explants denoted FB3. The demineralization of the extract was carried out by reconstituting a solution from freeze-dried powder with Milli-Q water, using dialysis bags (Spectrum, Spectra/Por with a cut-off at 0.5 kDa). The dissolved extract is dialyzed against a buffer of renewed Milli-Q water (<1 μS/cm) and maintained at ambient temperature with stirring until the conductivity is less than 1 μS/cm (2 micro siemens per centimeter correspond to approximately 1 milligram of salt dissolved per liter).

The same extraction method was used with the PCC limestone material supplied by the company VWR International SA. The total extract is used to treat a batch of explants denoted FB5, for 10 days.

A series of stainings and immunochemical labelings is carried out on the sections of skin explants after treatment of the three batches: FA1, FB3 and FB5, under the conditions described in examples 11 and 12 above.

The FA1 batch treated for 10 days with the Orgon organo-mineral extract obtained in this example showed, compared with the control batch, a moderate overexpression of neutral GAGs and of collagen type IV. This is interpreted as a stimulation of the activity of the dermal-epidermal junction since neutral GAGs are known to constitute the reservoir of growth factors at the dermal-epidermal junction (see table 13 below). These effects are sought after in the cosmetics industry.

The FB3 batch was treated with the organic fraction of the active agent concentrated during the dialysis by removal of the soluble trace elements. In the FB3 batch, an overexpression of neutral GAGs, of laminin-5 and of collagen types IV and III, and more clearly of collagen type I, were shown. The concentrated organic fraction, tested on the FB3 batch, clearly explains the stimulation of the dermal-epidermal junction discovered with the crude extract obtained in this example by means of the method of the invention. An overexpression of collagen I, which is an important component of the extracellular matrix of the papillary dermis, was also noted (see table 13 below).

The FB5 batch was treated with the extract obtained in this example, but from a limestone material, PCC (precipitated calcium carbonate), supplied by the company VWR International SA. A stimulation of neutral GAGs, of collagen IV and of laminin-5, which are all constituents of the dermal-epidermal junction, was surprisingly shown after 10 days of treatment (see table 13 below).

TABLE 13
Results obtained with the extracts obtained by means of the method of
the invention, compared with controls

| | |
|---|---|
| Extract | D5 |

| Batch | origin | Ki67 | G6PDH | Loricrin | Lekti |
|---|---|---|---|---|---|
| DA2 | OrgonW | [symbol] [symbol] | [symbol] | [symbol] | [symbol] [symbol] |
| DB6 | PCC | [symbol] | [symbol] [symbol] | [symbol] | [symbol] |
| | (VWR)W | | | | |
| DB2 | NacreW | [symbol] | [symbol] | [symbol] | [symbol] [symbol] |

D9/D10

| | | Coll I | Coll III | Coll IV | Lam 5 |
|---|---|---|---|---|---|
| Batch | Extract | [symbol] | [symbol] | [symbol] | [symbol] |
| | origin | | | | |
| DA2 | OrgonW | [symbol] | [symbol] | [symbol] [symbol] | [symbol] [symbol] |
| DB6 | PCC | [symbol] [symbol] | [symbol] | [symbol] | [symbol] [symbol] [symbol] |
| | (VWR)W | | | | |
| DB2 | NacreW | | | | |

Decrease:
slight: [symbol] ;
moderate: [symbol] [symbol] ;
quite clear: [symbol] [symbol] [symbol] ;
clear: [symbol] [symbol] [symbol] [symbol] .
Increase:
slight: [symbol] ;
moderate: [symbol] [symbol] ;
quite clear: [symbol] [symbol] [symbol] ;
clear: [symbol] [symbol] [symbol] [symbol] or
unchanged: [symbol] .

This example demonstrates that the fractionation of the extracts obtained with the method of the invention can make it possible to obtain amplified effects and/or effects targeted on a precise compartment or a precise function of the cells and/or physiological processes of the skin.

### LIST OF REFERENCES

(Ref 1) L Greger et al. “Mineral utilisation by rats fed various commercially available calcium supplements or milk.” J Nutr; 117: 717-24, 1987
(Ref 2) T. Chevalley et al. “Effect of calcium supplements on femoral bone mineral density and vertebrate fracture in Vitamin D depleted elderly patients”, Osteoporosis Int 4: 245-52, 1984
(Ref 3) Blumberg S. “Is coral calcium a safe and effective supplement?” J Am Diet Assoc September; 104(9): 1335-6
(Ref 4) T. Okano et al. “Bioavailability of calcium from oyster shell electrolysate and DL-Calcium lactate in vitamin D-replete or vitamin D-deficient rats.” Journal of Bone and Mineral Metabolism 11 (2): 23-32, 1993
(Ref 5) T. Miura “Calcium bioavailability of a total bone extract (TBE) and diets effects on bone metabolism in rats”, Biosci. Biotechnol. Biochem 62: 1307-12, 1998
(Ref 6) Y Takada “bioavailability of various dietary calcium compound. 2 Examination in vivo of various dietary compound>>, Snow brand milk products Co, Ltd Sapporo: 75-84
(Ref 7) A Schaafsmal et al. “Eggshell powder, a comparable or better source of calcium than purified calcium carbonate: piglet studies”, J Sci Food Agric 79: 1596-600, 1999
(Ref 8) E. Lafargue, F. Marquis, D. Pillot, “Rock-Eval 6 Applications in hydrocarbon exploration, production and soil contamination studies”, Oil & Gas Science and Technology, 1998. 53(4), 421-37
(Ref 9) Bellows C G, Heersche J N, Aubin J E. Bone Miner. Inorganic phosphate added exogenously or released from beta-glycerophosphate initiates mineralization of osteoid nodules in vitro. April; 17(1):15-29, 1992
(Ref 10) Bellows C G, Aubin J E, Heersche J N. Initiation and progression of mineralization of bone nodules formed in vitro: the role of alkaline phosphatase and organic phosphate Bone Miner. July; 14(1):27-40, 1991

1. A method for extracting organic and/or organomineral molecules from a limestone material, characterized in that it comprises bringing said limestone material into contact with a solution, said solution comprising: at least one organic solvent, water and at least one acid, said solution having a pH of between 0 and 5.

2. The method as claimed in claim 1, in which the organic solvent is chosen from the group comprising C1 to C6 alcohols, ethers and hydrocarbons.

3. The method as claimed in claim 2, in which the solvent is chosen from the group comprising ethanol, diethyl ether, dichloromethane, dimethylformamide (DMF), dimethyl sulfoxide (DMSO) and acetonitrile.

4. The method as claimed in claim 1, in which the acid(s) is (are) chosen from the group comprising inorganic acids and organic acids.

5. The method as claimed in claim 4, in which the acid is chosen from the group comprising hydrochloric acid, acetic acid, sulfuric acid, citric acid and ethylenediaminetetraacetic acid.

6. The method as claimed in claim 1, in which the concentration of said at least one organic solvent in said solution is between 1 and 99%.

7. The method as claimed in claim 1, in which the bringing into contact is carried out at a temperature of between 5 and 90° C.

8. The method as claimed in claim 1, in which said limestone material is crushed prior to bringing said material into contact with said solution.

9. The method as claimed in claim 8, in which said limestone material has an average particle size of between 0.5 and 500 μm.

10. The method as claimed in claim 1, in which the limestone material is a limestone rock.

11. The method as claimed in claim 10, in which the limestone rock is a limestone rock from France, chosen from the group comprising a rock from Orgon, France, stone from Euville, France, Saint Germain, France, Pagny sur Meuse, France, or Sainte Croix de Mareuil, France.

12. Organic and/or organomineral molecules that can be obtained by the method as claimed in claim 1.

13. A method for producing a cosmetic composition, which comprises implementing the method as claimed in claim 1 and mixing the organic and/or organomineral molecules in a cosmetically acceptable support.

14. A method for producing a pharmaceutical composition, which comprises implementing the method as claimed in claim 1 and mixing the organic and/or organomineral molecules in a pharmaceutically acceptable support.

15. A method as claimed in claim 14, in which the pharmaceutical composition is a medicament intended for increasing bone, cartilage or skin tissue regeneration.

16. The method as claimed in claim 14, in which the pharmaceutical composition is a medicament intended for the treatment of arthrosis, arthritis, osteoporosis or degenerative aging.
